(19) United States
(12) Patent Application Publication
(10) Pub. No.: US 2012/0232102 A1
(43) Pub. Date: Sep. 13, 2012

(54) Methods Of Administration And Treatment

(76) Inventor: Chun-fang Xu, Stevenage (GB)

(21) Appl. No.: 13/497,352

(22) PCT Filed: Sep. 29, 2010
(86) PCT No.: PCT/IB10/02690

§ 371 Date: Mar. 21, 2012

Related U.S. Application Data
(60) Provisional application No. 61/247,352, filed on Sep. 30, 2009.

(51) Int. Cl. A61K 31/506; A61P 35/00

(52) U.S. Cl. 514/272

(57) ABSTRACT

The present invention is directed to methods of administering pazopanib or pharmaceutically acceptable salts or solvates thereof as well as methods of treating cancer and age-related macular degeneration in patients in need thereof.

### FIELD OF THE INVENTION

The present invention relates to the administration of drug and its effects on patients with particular mutation(s) of the HFE gene.

### SUMMARY OF THE INVENTION

According to one aspect of the present invention, a method of administering a compound of formula (I) to a Caucasian patient in need thereof includes determining whether said patient has the TT genotype at the rs2858996 and/or rs707889 reference single nucleotide polymorphism, and if said patient does not have the TT genotype at the rs2858996 and/or rs707889 reference single nucleotide polymorphism, administering to said patient a compound

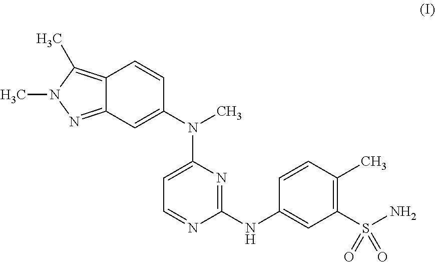

or a pharmaceutically acceptable salt or solvate thereof.

According to another aspect of the present invention, a method of prescribing a compound of formula (I) to a Caucasian patient in need thereof includes determining whether said patient has the TT genotype at the rs2858996 and/or rs707889 reference single nucleotide polymorphism, and if said patient does not have the TT genotype at the rs2858996 and/or rs707889 reference single nucleotide polymorphism, prescribing to said patient a compound of formula (I), or a pharmaceutically acceptable salt or solvate thereof.

According to still another aspect of the present invention, a method of treating cancer in a Caucasian patient in need thereof includes determining whether said patient has the TT genotype at the rs2858996 and/or rs707889 reference single nucleotide polymorphism, and if said patient does not have the TT genotype at the rs2858996 and/or rs707889 reference single nucleotide polymorphism, administering to said patient a compound of formula (I), or a pharmaceutically acceptable salt or solvate thereof.

According to another aspect of the present invention, a method of treating cancer in a Caucasian patient in need thereof, the patient having been previously genotyped as not having the TT genotype at the rs2858996 and/or rs707889 reference single nucleotide polymorphism, includes administering to the patient a compound of Formula (I), or a pharmaceutically acceptable salt or solvate thereof.

According to a further aspect of the present invention, a method of treating cancer in a Caucasian patient in need thereof includes administering to the patient a compound of formula (I), or a pharmaceutically acceptable salt or solvate thereof, and then determining whether the patient has a significant elevation in alanine aminostransferase.

According to yet another aspect of the present invention, a method of treating cancer in a Caucasian patient in need thereof includes administering to the patient a compound of formula (I) or a pharmaceutically acceptable salt or solvate thereof, and then determining whether said patient has the TT genotype at the rs2858996 and/or rs707889 reference single nucleotide polymorphism.

According to still another aspect of the present invention, a method of treating cancer in a Caucasian patient in need thereof includes determining whether said patient has the TT genotype at the rs2858996 and/or rs707889 reference single nucleotide polymorphism, administering to said patient a compound of formula (I), or a pharmaceutically acceptable salt or solvate thereof, and then determining whether said patient has a significant elevation in alanine aminostransferase.

According to a further aspect of the present invention, a method of treating age-related macular degeneration in a Caucasian patient in need thereof includes determining whether said patient has the TT genotype at the rs2858996 and/or rs707889 reference single nucleotide polymorphism, and if said patient does not have the TT genotype at the rs2858996 and/or rs707889 reference single nucleotide polymorphism, administering to said patient a compound of formula (I), or a pharmaceutically acceptable salt or solvate thereof.

According to another aspect of the present invention, a method of treating age-related macular degeneration in a Caucasian patient in need thereof, the patient having been previously genotyped as not having the TT genotype at the rs2858996 and/or rs707889 reference single nucleotide polymorphism, includes administering to the patient a compound of Formula (I), or a pharmaceutically acceptable salt or solvate thereof.

According to a further aspect of the present invention, a method of treating age-related macular degeneration in a Caucasian patient in need thereof includes administering to the patient a compound of formula (I), or a pharmaceutically acceptable salt or solvate thereof, and then determining whether the patient has a significant elevation in alanine aminostransferase.

According to yet another aspect of the present invention, a method of treating age-related macular degeneration in a Caucasian patient in need thereof includes administering to the patient a compound of formula (I) or a pharmaceutically acceptable salt or solvate thereof, and then determining whether said patient has the TT genotype at the rs2858996 and/or rs707889 reference single nucleotide polymorphism.

According to still another aspect of the present invention, a method of treating age-related macular degeneration in a Caucasian patient in need thereof determining whether said patient has the TT genotype at the rs2858996 and/or rs707889 reference single nucleotide polymorphism, administering to said patient a compound of formula (I), or a pharmaceutically acceptable salt or solvate thereof, and then determining whether said patient has a significant elevation in alanine aminostransferase.

### BRIEF DESCRIPTION OF THE DRAWINGS

FIG. 1 illustrates maximum alanine aminotransferase (ALT) times upper limit of normal (ULN) adjusted by baseline ALT times upper limit of normal for genotypes G,G, G,T, and T,T at the rs2858996 reference single nucleotide polymorphism;

FIG. 2 illustrates genotype proportion by trait status for genotypes G,G, G,T, and T,T at the rs2858996 reference single nucleotide polymorphism.

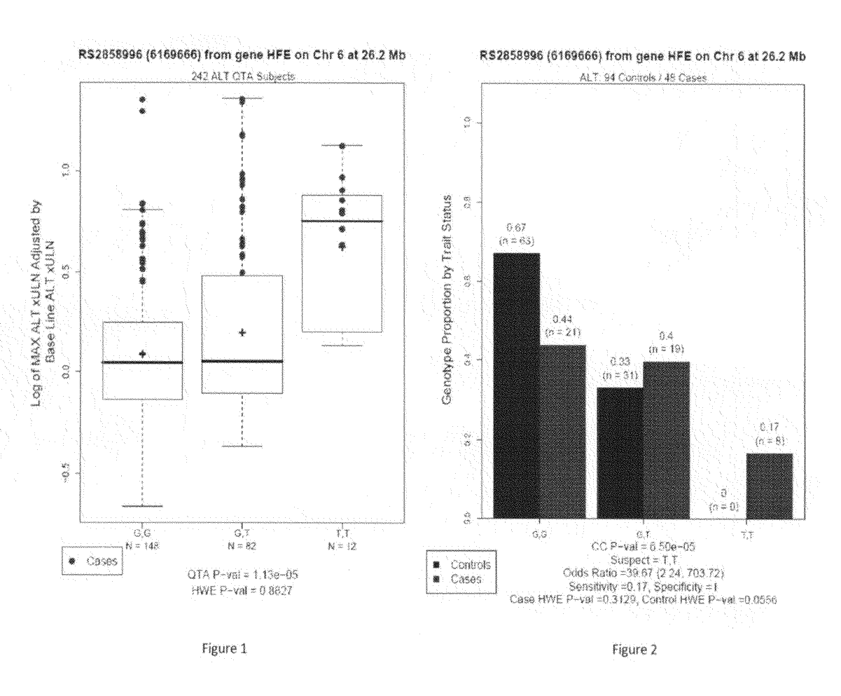

### DETAILED DESCRIPTION OF THE INVENTION

As used herein, the term “solvate” refers to a complex of variable stoichiometry formed by a solute (in this invention, compounds of formula (I), (II), (III), or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. In particular embodiments, the solvent used is water. One of ordinary skill in the art will readily appreciate how to determine if a solvate of compounds I, I′, and/or I″ will form and how to determine the composition of the solvate using standard solvate screening technology understood by those skilled in the art, for example.

The compound of formula (I):

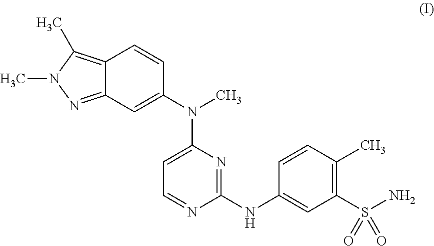

has the chemical name 5-[[4-[(2,3-dimethyl-2H-indazol-6-yl)methylamino]-2-pyrimidinyl]amino]-2-methylbenzenesulfonamide and is known by the generic name pazopanib.

In one aspect of the present invention, a method of administering a compound of formula (I) to a Caucasian patient in need thereof includes determining whether the patient has the TT genotype at the rs2858996 and/or rs707889 reference single nucleotide polymorphism, and if the patient does not have the TT genotype at the rs2858996 and/or rs707889 reference single nucleotide polymorphism, administering to the patient a compound of formula (I) or a pharmaceutically acceptable salt or solvate thereof.

While the rs2858996 reference single nucleotide polymorphism is understood to have the sequence:

TTACTGTACCTTAACCCTGAGTTTGC[G/T]TAGCTATCACTCACCAAT
TATGCAT

those skilled in the art will appreciate that polymorphisms which are similar to the [G/T] polymorphism shown in the sequence can also exist, namely [A/T] and [C/T]. When rs2858996 is used herein, it is meant to include the [G/T], [A/T], and [C/T] polymorphisms.

While the rs707889 reference single nucleotide polymorphism is understood to have the sequence:

GTTCCCCTTCATGTGATTCAAGCTCA[C/T]TCAGAAGAAACACAATGA
GACAAGA

those skilled in the art will appreciate that polymorphisms which are similar to the [C/T] polymorphism shown in the sequence can also exist, namely [A/T] and [G/T]. When rs2858996 is used herein, it is meant to include the [G/T], [A/T], and [C/T] polymorphisms.

The rs2858996 and rs707889 reference single nucleotide polymorphisms for which sequences are shown above can be detected using various oligonucleotides as will be understood by those skilled in the art, including:

| SNP | |
|---|---|
| | Oligo 1 Sequence (5′) |
| rs707889 | ACTTCGTCAGTAACGGACGCCCCTTCATGTGATTCAAGCTCAT |
| rs2858996 | ACTTCGTCAGTAACGGACTTACTGTACCTTAACCCTGAGTTTGCT |
| | Oligo 2 Sequence (5′) |
| rs707889 | GAGTCGAGGTCATATCGTGCCCCTTCATGTGATTCAAGCTCAC |
| rs2858996 | GAGTCGAGGTCATATCGTTTACTGTACCTTAACCCTGAGTTTGCG |
| | Oligo 3 Sequence (3′) |
| rs707889 | CAGAAGAAACACAATGAGACAAGACCTACCTGGGACGTATGGAACGTCTGCCTAT |
| | AGTGAGTC |
| rs2858996 | AGCTATCACTCACCAATTATGCACCTACCTGGGACGTATGGAACGTCTGCCTATA |
| | GTGAGTC |

According to another aspect of the invention, a method of prescribing a compound of formula (I) to a Caucasian patient in need thereof includes determining whether the patient has the TT genotype at the rs2858996 and/or rs707889 reference single nucleotide polymorphism, and if the patient does not have the TT genotype at the rs2858996 and/or rs707889 reference single nucleotide polymorphism, prescribing to the patient a compound of formula (I) or a pharmaceutically acceptable salt or solvate thereof.

In embodiments according to the various aspects of the present invention described herein, the determination of whether the patient has the TT genotype at the rs2858996 and/or rs707889 reference single nucleotide polymorphism includes determining whether the patient has the TT genotype at rs2858996 and rs707889. In other embodiments according to the aspects of the present invention described herein, the determination of whether the patient has the TT genotype at the rs2858996 and/or rs707889 reference single nucleotide polymorphism includes determining whether the patient has the TT genotype at rs2858996. In still other embodiments, the determination of whether the patient has the TT genotype at the rs2858996 and/or rs707889 reference single nucleotide polymorphism includes determining whether the patient has the TT genotype at rs707889.

In embodiments according to the various aspects of the present invention described herein, the determination of whether the patient has the TT genotype at the rs2858996 and/or rs707889 reference single nucleotide polymorphism includes testing the patient for the rs2858996 and/or rs707889 reference single nucleotide polymorphism. In other embodiments, the determination of whether the patient has the TT genotype at the rs2858996 and/or rs707889 reference single nucleotide polymorphism includes testing the patient for the rs2858996 and rs707889 reference single nucleotide polymorphisms. In still other embodiments, the patient is tested for the rs2858996 reference single nucleotide polymorphism. In yet other embodiments, the patient is tested for the rs707889 reference single nucleotide polymorphism. The testing of a patient to determine whether the patient has rs2858996 or rs707889 can be done by various methods as will be understood by those skilled in the art, for example as described in the Examples section below.

In embodiments according to the various aspects of the present invention described herein, the determination of whether the patient has the TT genotype at the rs2858996 and/or rs707889 reference single nucleotide polymorphism includes testing the patient for at least one single nucleotide polymorphism that is correlated with the rs2858996 and/or rs707889 reference single nucleotide polymorphism. In other embodiments, the determination of whether the patient has the TT genotype at the rs2858996 and/or rs707889 reference single nucleotide polymorphism includes testing the patient for at least one single nucleotide polymorphism that is correlated with the rs2858996 and rs707889 reference single nucleotide polymorphisms. In still other embodiments, determination of whether the patient has the TT genotype at the rs2858996 and/or rs707889 reference single nucleotide polymorphism includes testing the patient for at least one single nucleotide polymorphism that is correlated with the rs2858996 reference single nucleotide polymorphism. In yet other embodiments, determination of whether the patient has the TT genotype at the rs2858996 and/or rs707889 reference single nucleotide polymorphism includes testing the patient for at least one single nucleotide polymorphism that is correlated with the rs707889 reference single nucleotide polymorphism.

As used herein, a first reference single nucleotide polymorphism is correlated to a second single nucleotide polymorphism if detection of the first reference single nucleotide polymorphism, or a particular genotype of the first single nucleotide polymorphism, indicates that the individual would have the second reference single nucleotide polymorphism, or a particular genotype of the second reference single nuclear polymorphism, if the individual were to be tested for the second reference single nucleotide polymorphism or particular genotype thereof.

According to another aspect of the present invention, a method of treating cancer in a Caucasian patient in need thereof, the patient having been previously genotyped as not having the TT genotype at the rs2858996 and/or rs707889 reference single nucleotide polymorphism, includes administering to the patient a compound of Formula (I), or a pharmaceutically acceptable salt or solvate thereof.

In some embodiments according to this aspect of the present invention, the patient has been previously genotyped as not having the TT genotype at the rs2858996 and rs707889 reference single nucleotide polymorphisms. In other embodiments, the patient has been previously genotyped as not having the TT genotype at rs2858996. In still other embodiments, the patient has been previously genotyped as not having the TT genotype at rs707889.

According to still another aspect of the present invention, a method of treating cancer in a Caucasian patient in need thereof, the patient having previously been genotyped as not having at least one single nucleotide polymorphism that is correlated with the IT genotype of the rs2858996 and/or rs707889 reference single nucleotide polymorphism, includes administering to said patient a compound of Formula (I), or a pharmaceutically acceptable salt or solvate thereof.

In some embodiments according to this aspect of the present invention, the patient has been previously genotyped as not having at least one single nucleotide polymorphism that is correlated with the TT genotype of the rs2858996 and rs707889 reference single nucleotide polymorphisms. In other embodiments, the patient has been previously genotyped as not having at least one single nucleotide polymorphism that is correlated with the TT genotype of the rs2858996 reference single nucleotide polymorphisms. In still other embodiments, the patient has been previously genotyped as not having at least one single nucleotide polymorphism that is correlated with the TT genotype of the rs707889 reference single nucleotide polymorphisms.

In embodiments according to the various aspects of the present invention, the patient does not show significant elevation in alanine aminotransferase (ALT) after the administration of at least one dose of Formula I, or a pharmaceutically acceptable salt or solvate thereof, such as the compounds of Formula (I′) and (I″).

As used herein, the term “significant elevation in alanine aminotransferase” means≧3 times upper limit normal (ULN).

According to a further aspect of the present invention, a method of treating cancer in a Caucasian patient in need thereof includes administering to the patient a compound of formula (I), or a pharmaceutically acceptable salt or solvate thereof, and then determining whether the patient has a significant elevation in alanine aminostransferase.

If said patient has a significant elevation in alanine aminotransferase, some embodiments according to this aspect of the present invention further include determining whether said patient has the TT genotype at the rs2858996 and/or rs707889 reference single nucleotide polymorphism as described elsewhere herein.

Some embodiments according to this aspect of the present invention further include discontinuing treatment with a compound according to formula (I), or a pharmaceutically acceptable salt or solvate thereof.

According to yet another aspect of the present invention, a method of treating cancer in a Caucasian patient in need thereof includes administering to the patient a compound of formula (I) or a pharmaceutically acceptable salt or solvate thereof, and then determining whether said patient has the TT genotype at the rs2858996 and/or rs707889 reference single nucleotide polymorphism.

Some embodiments according to this aspect of the present invention further include discontinuing treatment with a compound according to formula (I), or a pharmaceutically acceptable salt or solvate thereof.

According to still another aspect of the present invention, a method of treating cancer in a Caucasian patient in need thereof determining whether said patient has the TT genotype at the rs2858996 and/or rs707889 reference single nucleotide polymorphism, administering to said patient a compound of formula (I), or a pharmaceutically acceptable salt or solvate thereof, and then determining whether said patient has a significant elevation in alanine aminostransferase.

If said patient has a significant elevation in alanine aminotransferase, some embodiments according to this aspect of the present invention further include discontinuing treatment with a compound according to formula (I), or a pharmaceutically acceptable salt or solvate thereof.

According to another aspect of the present invention, a method of screening a Caucasian human subject as an aid in predicting elevation in alanine aminotransferase (ALT) after administration of at least one dose of Formula I, or a pharmaceutically acceptable salt or solvate thereof, includes determining whether the patient has the TT genotype at the rs2858996 and/or rs707889 reference single nucleotide polymorphism, wherein the presence of at least one TT genotype indicates the subject is at increased risk for increased ALT after administration of Formula I, or a pharmaceutically acceptable salt or solvate thereof, such as a compound of formula (I′) or formula (I″).

According to still another aspect of the present invention, a method of screening a Caucasian human subject as an aid in predicting elevation in alanine aminotransferase (ALT) after administration of at least one dose of Formula I, or a pharmaceutically acceptable salt or solvate thereof, includes determining whether the patient has at least one single nucleotide polymorphism that is correlated with the TT genotype of the rs2858996 and/or rs707889 reference single nucleotide polymorphisms, wherein the presence of at least one single nucleotide polymorphism correlated with the TT genotype indicates the subject is at increased risk for increased ALT after administration of Formula I, or a pharmaceutically acceptable salt or solvate thereof, such as a compound of formula (I′) or formula (I″)

According to yet another aspect of the present invention, a method of identifying a Caucasian human subject at increased risk of experiencing increased alanine aminotransferase (ALT) greater than or equal to three time upper limit normal after administration of at least one dose of Formula I, or a pharmaceutically acceptable salt or solvate thereof, includes:
a. performing a genotyping technique on a biological sample from the subject to determine whether the subject has at least one single nucleotide polymorphism that is correlated with the TT genotype of the rs2858996 and/or rs707889;
b. detecting at least one single nucleotide polymorphism that is correlated with the TT genotype of the rs2858996 and/or rs707889; and
c. correlating the detection of at least one single nucleotide polymorphism that is correlated with the TT genotype of the rs2858996 and/or rs707889 with an increased risk of experiencing increased ALT greater than or equal to 3×ULN to at least one dose of Formula I, or a pharmaceutically acceptable salt thereof, compared to the risk if no single nucleotide polymorphism that is correlated with the TT genotype of the rs2858996 and/or rs707889 were detected.

In some embodiments, the biological sample is selected from the group consisting of cells, blood, blood components, urine and saliva.

The term “wild type” as is understood in the art refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild type polypeptide or polynucleotide, respectively. Included in the term mutant is Single Nucleotide Polymorphism (SNP) where a single base pair distinction exists in the sequence of a nucleic acid strand compared to the most prevalently found (wild type) nucleic acid strand. As used herein “genetic modification” or “genetically modified” refers to, but is not limited to, any suppression, substitution, deletion and/or insertion of one or more bases into DNA sequence(s). Also, as used herein “genetically modified” can refer to a gene encoding a polypeptide or a polypeptide having at least one deletion, substitution or suppression of a nucleic acid or amino acid, respectively.

Genetic mutations and/or SNPs can be identified by known methods. For example, wild type or SNPs can be identified by DNA amplification and sequencing techniques, DNA and RNA detection techniques, including, but not limited to Northern and Southern blot, respectively, and/or various biochip and array technologies. WT and mutant polypeptides can be detected by a variety of techniques including, but not limited to immunodiagnostic techniques such as ELISA and western Blot.

As used herein, the process of detecting an allele or polymorphism includes but is not limited to serologic and genetic methods. The allele or polymorphism detected may be functionally involved in affecting an individual's phenotype, or it may be an allele or polymorphism that is in linkage disequilibrium with a functional polymorphism/allele.

Polymorphisms/alleles are evidenced in the genomic DNA of a subject, but may also be detectable from RNA, cDNA or protein sequences transcribed or translated from this region, as will be apparent to one skilled in the art.

As is well known genetics, nucleotide and related amino acid sequences obtained from different sources for the same gene may vary both in the numbering scheme and in the precise sequence. Such differences may be due to numbering schemes, inherent sequence variability within the gene, and/or to sequencing errors. Accordingly, reference herein to a particular polymorphic site by number will be understood by those of skill in the art to include those polymorphic sites that correspond in sequence and location within the gene, even where different numbering/nomenclature schemes are used to describe them.

As used herein, “genotyping” a subject (or DNA or other biological sample) for a polymorphic allele of a gene(s) means detecting which allelic or polymorphic form(s) of the gene(s) or gene expression products (e.g., hnRNA, mRNA or protein) are present or absent in a subject (or a sample). Related RNA or protein expressed from such gene may also be used to detect polymorphic variation. As is well known in the art, an individual may be heterozygous or homozygous for a particular allele. More than two allelic forms may exist, thus there may be more than three possible genotypes. For purposes of the present invention, “genotyping” includes the determination of HLA alleles using suitable serologic techniques, as are known in the art. As used herein, an allele may be ‘detected’ when other possible allelic variants have been ruled out; e.g., where a specified nucleic acid position is found to be neither adenine (A), thymine (T) or cytosine (C), it can be concluded that guanine (G) is present at that position (i.e., G is ‘detected’ or ‘diagnosed’ in a subject). Sequence variations may be detected directly (by, e.g, sequencing) or indirectly (e.g., by restriction fragment length polymorphism analysis, or detection of the hybridization of a probe of known sequence, or reference strand conformation polymorphism), or by using other known methods.

As used herein, a “genetic subset” of a population consists of those members of the population having a particular genotype. In the case of a biallelic polymorphism, a population can potentially be divided into three subsets: homozygous for allele 1 (1,1), heterozygous (1,2), and homozygous for allele 2 (2,2). A ‘population’ of subjects may be defined using various criteria, e.g., individuals being treated with pazopanib or individuals with cancer.

As used herein, a subject that is “predisposed to” or “at increased risk of” a particular phenotypic response based on genotyping will be more likely to display that phenotype than an individual with a different genotype at the target polymorphic locus (or loci). Where the phenotypic response is based on a multi-allelic polymorphism, or on the genotyping of more than one gene, the relative risk may differ among the multiple possible genotypes.

“Genetic testing” (also called genetic screening) as used herein refers to the testing of a biological sample from a subject to determine the subject's genotype; and may be utilized to determine if the subject's genotype comprises alleles that either cause, or increase susceptibility to, a particular phenotype (or that are in linkage disequilibrium with allele(s) causing or increasing susceptibility to that phenotype).

“Linkage disequilibrium” refers to the tendency of specific alleles at different genomic locations to occur together more frequently than would be expected by chance. Alleles at given loci are in complete equilibrium if the frequency of any particular set of alleles (or haplotype) is the product of their individual population frequencies A commonly used measure of linkage disequilibrium is r:

$$r = \frac{{\hat{Δ}}_{AB}}{\sqrt{( {\tilde{π}}_{A} + {\hat{D}}_{A} ) ( {\tilde{π}}_{B} + {\hat{D}}_{B} )}}$$

$$where$$

$${\tilde{π}}_{A} = {\tilde{p}}_{A} ( 1 - {\tilde{p}}_{A} ) , \\ {\tilde{π}}_{B} = {\tilde{p}}_{B} ( 1 - {\tilde{p}}_{B} ) , \\ {\hat{D}}_{A} = {\tilde{P}}_{AA} - {\tilde{p}}_{A}^{2} , {\hat{D}}_{B} = {\tilde{P}}_{BB} - {\tilde{p}}_{B}^{2} \\ {\hat{Δ}}_{AB} = \frac{1}{n} {n}_{AB} - 2 {\tilde{p}}_{A} {\tilde{p}}_{B}$$

nr² has an approximate chi square distribution with 1 degree freedom for biallelic markers. Loci exhibiting an r such that nr² is greater than 3.84, corresponding to a significant chi-squared statistic at the 0.05 level, are considered to be in linkage disequilibrium (BS Weir 1996 Genetic Data Analysis II Sinauer Associates, Sunderland, Md.).

Alternatively, a normalized measure of linkage disequilibrium can be defined as:

$${D}_{AB}^{′} = \{ \begin{matrix}\frac{{D}_{AB}}{min ( {p}_{A} {p}_{B} , {p}_{a} {p}_{b} )} , & {D}_{AB} < 0 \\ \frac{{D}_{AB}}{min ( {p}_{A} {p}_{b} , {p}_{a} {p}_{B} )} , & {D}_{AB} > 0\end{matrix}$$

The value of the D′ has a range of −1.0 to 1.0. When statistically significant absolute D′ value for two markers is not less than 0.3 they are considered to be in linkage disequilibrium.

In certain embodiments, the salt of the compound of formula (I) is a hydrochloride salt. In a particular embodiment, the salt of the compound of formula (I) is a monohydrochloride salt as illustrated by formula (I′). The monohydrochloride salt of the compound of formula (I) has the chemical name 5-[[4-[(2,3-dimethyl-2H-indazol-6-yl)methylamino]-2-pyrimidinyl]amino]-2-methylbenzenesulfonamide monohydrochloride.

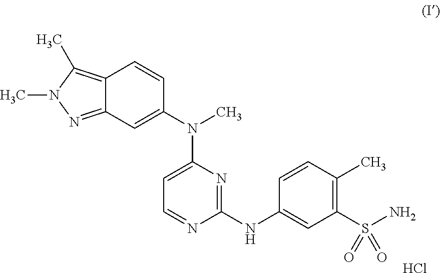

In other embodiments, the salt of the compound of formula (I) is a monohydrochloride monohydrate solvate of the compound of formula (I). The monohydrochloride monohydrate solvate of the compound of formula (I) has the chemical name 5-({4-[(2,3-dimethyl-2H-indazol-6-yl)methylamino]-2-pyrimidinyl}amino)-2-methylbenzenesulfonamide monohydrochloride monohydrate, as illustrated in formula (I″).

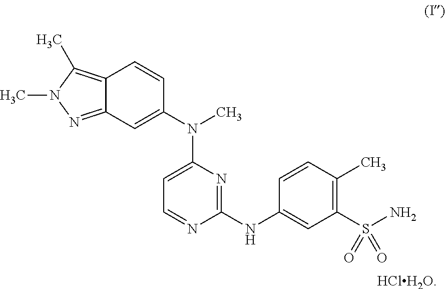

The free base, salts and solvates of the compound of formula (I) may be prepared, for example, according to the procedures of International Patent Application No. PCT/US01/49367 filed Dec. 19, 2001, and published as WO 02/059110 on Aug. 1, 2002, and International Patent Application No. PCT/US03/19211 filed Jun. 17, 2003, and published as WO 03/106416 on Dec. 24, 2003, or according to the methods provided herein.

As used herein, the term “pharmaceutically acceptable salts” may comprise acid addition salts derived from a nitrogen on a substituent in the compound of formula (I). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate.

While it is possible that, the compound of formula (I), as well as pharmaceutically acceptable salts and solvates thereof, may be administered as the raw chemical, it is also possible to present the active ingredient as a pharmaceutical composition. Accordingly, embodiments of the invention further provide pharmaceutical compositions, which include therapeutically effective amounts of compounds of the formula (I) and pharmaceutically acceptable salts and solvates thereof, and one or more pharmaceutically acceptable carriers, diluents, or excipients. The compound of the formula (I) and salts and solvates thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or pharmaceutically acceptable salts or solvates thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 800 mg, of a compound of the formula (I) depending on the condition being treated, the route of administration and the age, weight and condition of the patient. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additives such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compound of formula (I) and pharmaceutically acceptable salts and solvates thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compound of formula (I) and pharmaceutically acceptable salts and solvates thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent. Eye-drop formulations are described further herein below. In some embodiments, the eye-drop formulation includes from a lower limit of 1, 2, 3, 4, 5, 6, 7, or 8 and an upper limit of 2, 3, 4, 5, 6, 7, 8, 9, or 10 mg of a compound of formula (I) or a pharmaceutically acceptable salt or solvate thereof per ml. In some embodiments, the eye-drop formulation includes 2 mg of a compound of formula (I) or a pharmaceutically acceptable salt or solvate thereof per ml. In other embodiments, the eye-drop formulation includes 5 mg of a compound of formula (0 or a pharmaceutically acceptable salt or solvate thereof per ml.

Pharmaceutical formulations for topical administration to the eye may be presented in unit dose forms containing a predetermined amount of active ingredient per unit dose. Such a unit may contain, for example, 1 μg to 1 g, such as 5 μg to 500 μg, 10 μg-250 μg, 0.5 mg to 700 mg, 2 mg to 350 mg, or 5 mg to 100 mg of a compound of formula (I) or pharmaceutically acceptable salts or solvates thereof depending on the condition being treated, the route of administration and the age, weight and condition of the patient, or pharmaceutical formulations may be presented in unit dose forms containing a predetermined amount of active ingredient per unit dose. In certain embodiments, the unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Suitable routes for ocular administration include extraocular and intraocular (including, for example, intravitreal, subretinal, subscleral, intrachoroidal, and subconjuctival). It will be appreciated that the preferred route may vary with, for example, the condition of the recipient.

For treatments of the eye, the pharmaceutical formulations may also be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Formulations to be administered to the eye will have ophthalmically compatible pH and osmolality. One or more ophthalmically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions. Embodiments of pharmaceutical formulations suitable for ocular administration include the following:

| | Quantity (mg/ml) | | | |
|---|---|---|---|---|
| | Product Strength | | | |
| Component | Placebo | 2 mg/ml | 5 mg/ml | Function |
| Pazopanib | NA | 2.167 | 5.417 | Active |
| Monohydrochloride | | | | |
| B-cyclodextrin | 70.00 | 70.00 | 70.00 | Solubility |
| sulfobutylether | | | | Enhancer |
| (Captisol) | | | | |
| Monobasic Sodium | 3.450 | 3.450 | 3.450 | Buffering |
| Phosphate | | | | Agent |
| Sodium Chloride | 1.685 | 1.685 | 1.461 | Tonicity |
| | | | | Modifier |
| Water for injection | q.s. | q.s. | q.s. | Solvent |
| Hydrochloric Acid | As needed | As needed | As needed | pH Adjustment |
| Sodium Hydroxide | As needed | As needed | As needed | pH Adjustment |

These formulations are presented as a preservative free, single use eye drop formulations. Current fill per container is 0.45 mL with a drop weight of ˜40 μL. Density of solution is 1.03 mg/mL. Osmolality is ˜290 mOs m/kg. The pH of the formulations is 5. These formulations were used in obtaining the results detailed in the Biological section herein. These formulations can be modified to have a pH down to a value of 4. These formulations can also be modified to have a Captisol concentration up to 10% w/v.

In some embodiments of the present invention, the pharmaceutical formulations are adapted for intraocular administration by means of intraocular injection or other device for ocular delivery. Examples of ocular devices that may be used in the methods of the invention include periocular or intravitreal devices, contact lenses and liposomes. See, for example, U.S. Pat. Nos. 3,416,530; 3,828,777; 4,014,335; 4,300,557; 4,327,725; 4,853,224; 4,946,450; 4,997,652; 5,147,647; 5,164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146,366; 6,251,090; 6,299,895; 6,331,313; 6,416,777; 6,649,184; 6,719,750; 6,660,960; and U.S. Patent Publication Nos. 2003/0064088, 2004/0247645, and, 2005/0113806; each of which is herein incorporated by reference for purposes of their teachings of optical devices.

The ocular delivery device may be designed for the controlled release of one or more therapeutic agents with multiple defined release rates and sustained dose kinetics and permeability. Controlled release may be obtained through the design of polymeric matrices incorporating different choices and properties of biodegradable/bioerodable polymers (e.g. poly(ethylene vinyl) acetate (EVA), superhydrolyzed PVA), hydroxyalkyl cellulose (HPC), methylcellulose (MC), hydroxypropyl methyl cellulose (HPMC), polycaprolactone, poly(glycolic) acid, poly(lactic) acid, polyanhydride, of polymer molecular weights, polymer crystallinity, copolymer ratios, processing conditions, surface finish, geometry, excipient addition and polymeric coatings that will enhance drug diffusion, erosion, dissolution and osmosis.

Formulations for drug delivery using ocular devices may combine one or more active agents and adjuvants appropriate for the indicated route of administration. For example, the active agents may be admixed with any pharmaceutically acceptable excipient, lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, tableted or encapsulated for conventional administration. Alternatively, the compounds may be dissolved in polyethylene glycol, propylene glycol, carboxymethyl cellulose colloidal solutions, ethanol, corn oil, peanut oil, cottonseed oil, sesame oil, tragacanth gum, and/or various buffers. The compounds may also be mixed with compositions of both biodegradable and non-biodegradable polymers, and a carrier or diluent that has a time delay property. Representative examples of biodegradable compositions can include albumin, gelatin, starch, cellulose, dextrans, polysaccharides, poly (D,L-lactide), poly (D,L-lactide-co-glycolide), poly (glycolide), poly (hydroxybutyrate), poly (alkylcarbonate) and poly (orthoesters) and mixtures thereof. Representative examples of non-biodegradable polymers can include EVA copolymers, silicone rubber and poly (methylacrylate), and mixtures thereof.

Pharmaceutical compositions for ocular delivery also include in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein is includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig (2005) Adv. Drug Deliv. Rev. 3; 57:1595-639, herein incorporated by reference for purposes of its teachings of examples of polymers for use in ocular drug delivery.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of formula (I) or a pharmaceutically acceptable salt or solvate thereof will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of formula (I) or a salt or solvate thereof for the treatment of a cancerous condition such as those described herein will generally be in the range of 0.1 to 100 mg/kg body weight of recipient (mammal) per day and more usually in the range of 1 to 12 mg/kg body weight per day. Thus, for a 70 kg adult mammal, the actual amount per day would usually be from 70 to 840 mg and this amount may be given in a single dose per day or more usually in a number (such as two, three, four, five or six) of sub-doses per day such that the total daily dose is the same. An effective amount of a salt or solvate thereof may be determined as a proportion of the effective amount of the compound of formula (I) per se. It is envisaged that similar dosages would be appropriate for treatment of the other conditions referred to above.

The compound of formula (I) and pharmaceutically acceptable salts and solvates thereof may be employed alone or in combination with other therapeutic agents for the treatment of the above-mentioned conditions. In particular, in anti-cancer therapy, combination with other chemotherapeutic, hormonal or antibody agents is envisaged as well as combination with surgical therapy and radiotherapy. Combination therapies according to the present invention thus comprise the administration of a compound of formula (I) or a pharmaceutically acceptable salt or solvate thereof, or a physiologically functional derivative thereof, and the use of at least one other cancer treatment method. Preferably, combination therapies according to the present invention comprise the administration of a compound of formula (I) or a pharmaceutically acceptable salt or solvate thereof, or a physiologically functional derivative thereof, and at least one other pharmaceutically active agent, preferably an anti-neoplastic agent. The compound of formula (I) or a pharmaceutically acceptable salt or solvate thereof and the other pharmaceutically active agent(s) may be administered together or separately and, when administered separately this may occur simultaneously or sequentially in any order. The amounts of the compound of formula (I) or pharmaceutically acceptable salt or solvate thereof and the other pharmaceutically active agent(s) and the relative timings of administration will be selected in order to achieve the desired combined therapeutic effect.

The compound of Formula (I) or pharmaceutically acceptable salts or solvates thereof and at least one additional cancer treatment therapy may be employed in combination concomitantly or sequentially in any therapeutically appropriate combination with such other anti-cancer therapies. In one embodiment, the other anti-cancer therapy is at least one additional chemotherapeutic therapy including administration of at least one anti-neoplastic agent. The administration in combination of a compound of formula (I) or pharmaceutically acceptable salts or solvates thereof with other anti-neoplastic agents may be in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein one anti-neoplastic agent is administered first and the other second or vice versa. Such sequential administration may be close in time or remote in time.

Anti-neoplastic agents may induce anti-neoplastic effects in a cell-cycle specific manner, i.e., are phase specific and act at a specific phase of the cell cycle, or bind DNA and act in a non cell-cycle specific manner, i.e., are non-cell cycle specific and operate by other mechanisms.

Anti-neoplastic agents useful in combination with the compound of formula (I) or pharmaceutically acceptable salts or solvates thereof include the following:

(1) cell cycle specific anti-neoplastic agents including, but not limited to, diterpenoids such as paclitaxel and its analog docetaxel; vinca alkaloids such as vinblastine, vincristine, vindesine, and vinorelbine; epipodophyllotoxins such as etoposide and teniposide; fluoropyrimidines such as 5-fluorouracil and fluorodeoxyuridine; antimetabolites such as allopurinol, fludurabine, methotrexate, cladrabine, cytarabine, mercaptopurine and thioguanine; and camptothecins such as 9-amino camptothecin, irinotecan, CPT-11 and the various optical forms of 7-(4-methylpiperazino-methylene)-10,11-ethylenedioxy-20-camptothecin;

(2) cytotoxic chemotherapeutic agents including, but not limited to, alkylating agents such as melphalan, chlorambucil, cyclophosphamide, mechlorethamine, hexamethylmelamine, busulfan, carmustine, lomustine, and dacarbazine; anti-tumour antibiotics such as doxorubicin, daunomycin, epirubicin, idarubicin, mitomycin-C, dacttinomycin and mithramycin; and platinum coordination complexes such as cisplatin, carboplatin, and oxaliplatin; and

(3) other chemotherapeutic agents including, but not limited to, anti-estrogens such as tamoxifen, toremifene, raloxifene, droloxifene and iodoxyfene; progestrogens such as megestrol acetate; aromatase inhibitors such as anastrozole, letrazole, vorazole, and exemestane; antiandrogens such as flutamide, nilutamide, bicalutamide, and cyproterone acetate; LHRH agonists and antagagonists such as goserelin acetate and luprolide, testosterone 5α-dihydroreductase inhibitors such as finasteride; metalloproteinase inhibitors such as marimastat; antiprogestogens; urokinase plasminogen activator receptor function inhibitors; cyclooxygenase type 2 (COX-2) inhibitors such as celecoxib; other angiogenic inhibiting agents such as VEGFR inhibitors other than those described herein and TIE-2 inhibitors; growth factor function inhibitors such as inhibitors of the functions of hepatocyte growth factor; erb-B2, erb-B4, epidermal growth factor receptor (EGFr), platelet derived growth factor receptor (PDGFr), vascular endothelial growth factor receptor (VEGFR) other than those described in the present invention, and TIE-2; and other tyrosine kinase inhibitors such as cyclin dependent inhibitors such as CDK2 and CDK4 inhibitors.

The compound of formula (I) and pharmaceutically acceptable salts and solvates thereof are believed to have anticancer activity as a result of inhibition of the protein kinase VEGFR2 and its effect on selected cell lines whose growth is dependent on VEGFR2 protein kinase activity.

The present invention thus also provides for administration of a compound of formula (I) or pharmaceutically acceptable salts or solvates thereof for use in medical therapy of a Caucasian patient in need thereof in which the patient does not have the TT genotype at the rs2858996 and/or rs707889 reference single nucleotide polymorphism, and particularly in the treatment of such patients of disorders mediated by inappropriate VEGFR2 activity.

The inappropriate VEGFR2 activity referred to herein is any VEGFR2 activity that deviates from the normal VEGFR2 activity expected in a particular mammalian subject. Inappropriate VEGFR2 activity may take the form of, for instance, an abnormal increase in activity, or an aberration in the timing and or control of VEGFR2 activity. Such inappropriate activity may result then, for example, from overexpression or mutation of the protein kinase or ligand leading to inappropriate or uncontrolled activation of the receptor. Furthermore, it is also understood that unwanted VEGFR2 activity may reside in an abnormal source, such as a malignancy. That is, the level of VEGFR2 activity does not have to be abnormal to be considered inappropriate, rather the activity derives from an abnormal source. In a like manner, the inappropriate angiogenesis referred to herein is any angiogenic activity that deviates from the normal angiogenic activity expected in a particular mammalian subject. Inappropriate angiogenesis may take the form of, for instance, an abnormal increase in activity, or an aberration in the timing and or control of angiogenic activity. Such inappropriate activity may result then, for example, from overexpression or mutation of a protein kinase or ligand leading to inappropriate or uncontrolled activation of angiogenesis. Furthermore, it is also understood that unwanted angiogenic activity may reside in an abnormal source, such as a malignancy. That is, the level of angiogenic activity does not have to be abnormal to be considered inappropriate, rather the activity derives from an abnormal source.

Embodiments of the present invention are directed to methods of regulating, modulating, or inhibiting VEGFR2 for the prevention and/or treatment of disorders related to unregulated VEGFR2 activity in Caucasian patients in need thereof who do not have the TT genotype at the rs2858996 and/or rs707889 reference single nucleotide polymorphism. In particular, the compound of formula (I) and its pharmaceutically acceptable salts and solvates thereof can also be used in the treatment of certain forms of cancer. Furthermore, the compound of formula (I) and its pharmaceutically acceptable salts and solvates thereof can be used to provide additive or synergistic effects with certain existing cancer chemotherapies and radiation, and/or be used to restore effectiveness of certain existing cancer chemotherapies and radiation.

In one aspect of the present invention, a method of treating cancer in a Caucasian patient in need thereof includes determining whether the patient has the TT genotype at the rs2858996 and/or rs707889 reference single nucleotide polymorphism, and if the patient does not have the TT genotype at the rs2858996 and/or rs707889 reference single nucleotide polymorphism, administering to the patient a compound of formula (I), or a pharmaceutically acceptable salt or solvate thereof.

In embodiments according to the various aspects of the present invention, the cancer is selected from the group consisting of colon cancer, breast cancer, renal cell carcinoma, melanoma, lung cancer including non-small cell lung cancer and adenocarcinoma, gastric cancer, colorectal cancer, neuroendocrine cancer, thyroid cancer, head and neck cancer, brain cancer, cervical cancer, bladder cancer, esophageal cancer, pancreatic cancer, prostate cancer, mesothelioma, liver-hepatobiliary cancer, multiple myeloma, leukemia, thyroid cancer including Hurthle cell, muscle sarcoma (leiomyosarcoma) and bone sarcoma (chonrosarcoma).

As used herein, “treatment” means any manner in which one or more symptoms associated with the disorder are beneficially altered. Accordingly, the term includes healing or amelioration of a symptom or side effect of the disorder or a decrease in the rate of advancement of the disorder.

The compound of formula (I) and its pharmaceutically acceptable salts and solvates thereof are also useful in the treatment of one or more diseases afflicting Caucasian patients who do not have the TT genotype at the rs2858996 and/or rs707889 reference single nucleotide polymorphism, where the diseases are characterized by cellular proliferation in the area of disorders associated with neo-vascularization and/or vascular permeability including blood vessel proliferative disorders including arthritis and restenosis; fibrotic disorders including hepatic cirrhosis and atherosclerosis; mesangial cell proliferative disorders include glomerulonephritis, diabetic nephropathy, malignant nephrosclerosis, thrombotic microangiopathy syndromes, proliferative retinopathies, organ transplant rejection and glomerulopathies; and metabolic disorders include psoriasis, diabetes mellitus, chronic wound healing, inflammation and neurodegenerative diseases.

Embodiments of the present invention provides a method of treatment of a Caucasian patient suffering from a disorder mediated by inappropriate VEGFR2 activity, including susceptible malignancies, which includes determining whether the patient has the TT genotype at the rs2858996 and/or rs707889 reference single nucleotide polymorphism; and if the patient does not have the TT genotype at the rs2858996 or rs707889 reference single nucleotide polymorphism, administering to the patient a compound of formula (I) or a pharmaceutically acceptable salt or solvate thereof. In one embodiment, the disorder is cancer as described in more detail above.

Further embodiments of the present invention provides a method of treatment of a Caucasian patient suffering from cancer which includes determining whether said subject has the TT genotype at the rs2858996 and/or rs707889 reference single nucleotide polymorphism; and if the patient does not have the TT genotype at the rs2858996 and/or rs707889 reference single nucleotide polymorphism, administering to the patient a compound of formula (I) or a pharmaceutically acceptable salt or solvate thereof. In one embodiment, the disorder is cancer as described in more detail above.

A further aspect of the present invention provides the use of a compound of formula (I), or a pharmaceutically acceptable salt or solvate thereof in the preparation of a medicament for the treatment of a disorder characterized by inappropriate VEGFR2 activity. In one embodiment, the disorder is cancer as described in more detail above.

A further aspect of the present invention provides the use of a compound of formula (I) or a pharmaceutically acceptable salt or solvate thereof in the preparation of a medicament for the treatment of cancer and malignant tumours.

In other embodiments, the administration of the compound of formula (I) or pharmaceutically acceptable salts or solvates thereof can include administering the compound to a Caucasian patient who does not have the TT genotype at the rs2858996 and/or rs707889 reference single nucleotide polymorphism in combination with agents which inhibit growth factor receptor function to a patient for treatment of a disorder mediated by inappropriate VEGFR2 activity, for instance in the treatment of cancer. Such growth factor receptors include, for example, EGFR, PDGFR, erbB2, erbB4, VEGFR, and/or TIE-2. Growth factor receptors and agents that inhibit growth factor receptor function are described, for instance, in Kath, John C., Exp. Opin. Ther. Patents (2000) 10(6):803-818 and in Shawver et al DDT Vol 2, No. 2 Feb. 1997.

The compound of formula (I) or pharmaceutically acceptable salts or solvates thereof and the agent for inhibiting growth factor receptor function may be employed in combination concomitantly or sequentially in any therapeutically appropriate combination. The combination may be employed in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein one is administered first and the other second or vice versa. Such sequential administration may be close in time or remote in time.

In another aspect of the present invention, a method of treating a disorder in a Caucasian patient, the disorder being mediated by inappropriate angiogenesis, includes determining whether the patient has the TT genotype at the rs2858996 and/or rs707889 reference single nucleotide polymorphism, and if the patient does not have the TT genotype at the rs2858996 and/or rs707889 reference single nucleotide polymorphism, administering to the patient a compound of formula (I) or a pharmaceutically acceptable salt or solvate thereof. In one embodiment, the inappropriate angiogenic activity is due to at least one of inappropriate VEGFR1, VEGFR2, VEGFR3, or PGDFR alpha, beta and c-kit activity. In another embodiment, the inappropriate angiogenesis is due to inappropriate VEGFR2 and PGDFR alpha, beta and c-kit activity. In a further embodiment, the method further includes administering a PGDFR alpha, beta and c-kit inhibitor along with the compound of formula (I) or pharmaceutically acceptable salts or solvates thereof. Preferably the disorder is cancer as described in more detail above.

In another aspect of the present invention, there is provided the use of a compound of formula (I) or a pharmaceutically acceptable salt or solvate thereof in the preparation of a medicament for use in treating a disorder in a Caucasian patient who does not have the TT genotype at the rs2858996 and/or rs707889 reference single nucleotide polymorphism, the disorder being characterized by inappropriate angiogenesis. In one embodiment, the inappropriate angiogenic activity is due to at least one of inappropriate VEGFR1, VEGFR2, VEGFR3 or PGDFR alpha, beta and c-kit activity. In another embodiment, the inappropriate angiogenic activity is due to inappropriate VEGFR2 and PGDFR alpha, beta and c-kit activity. In a further embodiment, the use further includes use of a PGDFR alpha, beta and c-kit inhibitor to prepare said medicament.

The combination of a compound of formula (I) or a pharmaceutically acceptable salt or solvate thereof with a TIE-2 inhibitor may be employed in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein one is administered first and the other second or vice versa. Such sequential administration may be close in time or remote in time.

While the foregoing aspects according to the present invention have been described with respect to methods of treating cancer, it is also to be understood that aspects of the present invention include similar aspects directed to methods of treating age-related macular degeneration.

In some embodiments of methods according to these aspects of the present invention, the age-related macular degeneration is wet age-related macular degeneration. In other embodiments, the age-related macular degeneration is dry age-related macular degeneration. In still other embodiments, the age-related macular degeneration is late stage age-related macular degeneration.

The methods according to these aspects of the present invention may also be employed in combination with other methods for the treatment of ocular neovascular disorders. In some embodiments, the methods of the invention encompass a combination therapy in which a compound of formula (I) or a pharmaceutically acceptable salt or solvate thereof is administered in conjunction with one or more additional therapeutic agents for the treatment of neovascular disorders. Non-limiting examples of additional therapeutic agents that may be used in a combination therapy include pegaptanib, ranibizumab, bevacizumab, VEGF-TRAP, PKC412, nepafenac, and integrin receptor antagonists (including vitronectin receptor agonists). See, for example, Takahashi et al. (2003) Invest. Ophthalmol. Vis. Sci. 44: 409-15, Campochiaro et al. (2004) Invest. Ophthalmol. Vis. Sci. 45:922-31, van Wijngaarden et al. (2005) JAMA 293:1509-13, U.S. Pat. No. 6,825,188 to Callahan et al., and U.S. Pat. No. 6,881,736 to Manley et al.; each of which is herein incorporated by reference for their teachings regarding these compounds.

Where a combination therapy is employed, the therapeutic agents may be administered together or separately. The same means for administration may be used for more than one therapeutic agent of the combination therapy; alternatively, different therapeutic agents of the combination therapy may be administered by different means. When the therapeutic agents are administered separately, they may be administered simultaneously or sequentially in any order, both close and remote in time. The amounts of the compound of formula (I) or a pharmaceutically acceptable salt or solvate thereof and/or the other pharmaceutically active agent or agents and the relative timings of administration will be selected in order to achieve the desired combined therapeutic effect.

The amount of administered or prescribed compound according to these aspects of the present invention will depend upon a number of factors including, for example, the age and weight of the patient, the precise condition requiring treatment, the severity of the condition, the nature of the formulation, and the route of administration. Ultimately, the amount will be at the discretion of the attendant physician.

In some embodiments according to these aspects of the present invention, the total amount of the compound of formula (I) or a pharmaceutically acceptable salt or solvate thereof administered or prescribed to be administered per day can be 1 μg to 10 mg. In other embodiments, such amount can be 5 μg to 500 μg. In still other embodiments, such amount can be 10 μg-250 μg. In some embodiments, the compound of formula (I) or a pharmaceutically acceptable salt or solvate thereof is administered or prescribed to be administered one, two, three, four, or more times per day. In some embodiments, the compound of formula (I) or a pharmaceutically acceptable salt or solvate thereof is administered or prescribed to be administered by administering one, two, three, four or more drops of a suitable pharmaceutical formulation one, two, three, four, or more times per day. In some embodiments, the suitable pharmaceutical formulation comprises between a lower limit of 1, 2, 3, 4, 5, 6, 7, 8, or 9 and an upper limit of 2, 3, 4, 5, 6, 7, 8, 9, or 10 mg of a compound of formula (I) or a pharmaceutically acceptable salt or solvate thereof per ml.

The following examples are intended for illustration only and are not intended to limit the scope of the invention in any way.

### EXAMPLES

Objective:

To evaluate if genetic markers are associated with elevation of alanine aminotransferase in subjects treated with pazopanib.

Methodology:

Blood samples were collected for pharmacogenetic (PGx) study. DNA extraction was performed by using either the Qiagen QiaAmp column purification or Gentra Puregene protocols (Qiagen QIAamp® DNA Blood Midi/Maxi Handbook 01/2005, Qiagen Gentra Puregene Handbook Second Edition 09/2007).

Genetic markers in the HFE gene were analyzed. Genotyping was conducted by GoldenGate genotyping: a genotyping method developed by Illumina that uses a discriminatory DNA polymerase and ligase to interrogate up to 1536 SNP loci simultaneously (Fan, J B et al. 2004. Highly parallel SNP genotyping. Cold Spring Harbor Symposia on Quantitative Biology68, 69-78).

Number of Subjects:

Summary of the number of subjects in the intent to treat (ITT) and PGx analysis populations:

| ITT Population | PGx Analysis population¹ |
|---|---|
| 9 | 6 |
| 35 | 22 |
| 75 | 33 |
| 26 | 24 |
| 225 | 164 |
| 435 | 181 |

Sample Size Considerations:

The testing of PGx hypotheses was not the primary goal of the underlying clinical studies upon which this analysis was based; therefore, the analysis did not benefit from prospective sample size calculations or randomization schemes for the genetic markers. The results were thus interpreted as exploratory.

Criteria for Evaluation:

On-treatment ALT was assessed. A comparison of interest was differences in values of ALT between different genotype groups for the genetic marker in HFE gene.

Statistical Methods:

To minimize the effect of study design and population structure on the conclusions drawn from the PGx analysis, a separate PGx evaluation was performed for each Race/Ethnicity group within each clinical study. Power estimates of the PGx analysis populations showed only the White (or Caucasian) of size n=116 and n=130 had sufficient sample size to detect a significant statistical association for a major genetic effect. Therefore, inferential analyses were performed using White subjects from a first study in the primary analysis and White subjects from a second study in the confirmatory analysis. Statistical analyses were also performed for confirmed markers using White subjects combined from both studies.

Genotypes of significant markers (p≦0.01) identified in analysis of the White subjects from the first and then the second study were summarized for subjects in the other Race/Ethnicity clusters and other clinical studies where a formal inferential statistical analysis was not performed due to the limited sample sizes.

Quantitative trait analysis (QTA) and case-control (CC) analysis were applied to assess the association of genotypes with pazopanib induced ALT for White subjects. In the QTA, an analysis of covariance model, including terms for genotype and baseline, was used to assess the main effect of genotype on ALT. Maximum on-treatment and baseline ALT×Upper Limit of Normal (ULN) values were log₁₀—transformed prior to the QTA analysis. A permutation P value was also calculated to account for potential violations in the model assumptions and correct for a possible inflation of the type I error.

In case-control analysis, Fisher's Exact Test (FET) was used to examine the effect of genotypes on the case-control status for ALT. An ‘ALT case’ was defined as any patient exposed to pazopanib who had one or more on-treatment ALT measurements of 3.0×ULN or greater; whilst an ‘ALT control’ was defined as any patient exposed to pazopanib who had all on-treatment ALT measurements within the normal range (1×ULN or less).

A Hardy-Weinberg equilibrium (HWE) analysis was applied to test if the distribution of the observed genotypes in White subjects deviated from that expected according to the Hardy-Weinberg Principle.

Linkage disequilibrium between pairs of markers was evaluated to assess correlations between genetic markers.

### SUMMARY

Genetic markers in the HFE gene were found to be associated with ALT elevation in the primary PGx analysis using White subjects from the first study (p≦0.01). These genetic markers were evaluated in the confirmatory analysis in White subjects from the second study. Two of these markers (rs2858996 and rs707889) in the HFE gene were found to be significantly associated with ALT elevation in the second study (permutation QTA and FET p≦0.01).

No evidence of deviation from HWE was observed for these markers in either clinical study (p>0.49). Since these two markers were statistically highly correlated (r²=1.0 for the subjects from one study and r²=0.98 for the subjects from the other study), the analysis results for one of the markers (rs2858996) are summarized.

TABLE 1
Main Effect of HFE Genotypes from QTA on Maximum on-
Treatment ALTxULN (log₁₀) in Whites, First Study

| | | | | Genotype LS | |
|---|---|---|---|---|---|
| | | | | Mean Maximum | 95% CI of |
| Chromosome | RS | QTA | Genotype | on Treatment | Genotype |
| position (bp) | Number | P-Value | (N) | ALTxULN (SE) | LS Mean |
| 6: 026202005 | rs2858996 | 0.0058 | G, G (74) | 0.12 (0.04) | (0.03, 0.20) |
| | | | G, T (35) | 0.24 (0.06) | (0.11, 0.36) |
| | | | T, T (6) | 0.59 (0.15) | (0.30, 0.89) |

TABLE 2
Main Effect of HFE Genotypes from Case Control Analysis of ALT Elevation in
Whites, First Study

| RS | FET | Genotype | Proportion of | Proportion of | Suspect | Odds Ratio |
|---|---|---|---|---|---|---|
| Number | P-Value | (N) | (N) Cases | (N) Controls | Genotype | (95% CI) |
| rs2858996 | 0.0138 | G, G (42) | 0.48 (13) | 0.73 (29) | T, T | 15.5 (0.8, 301.0) |
| | | G, T (21) | 0.37 (10) | 0.28 (11) | | |
| | | T, T (4) | 0.15 (4) | 0.00 (0) | | |

TABLE 3
Main Effect of HFE Genotypes from QTA on Maximum on-
Treatment ALTxULN (log₁₀) in Whites, Second Study

| | | | | Genotype LS | |
|---|---|---|---|---|---|
| | | | | Mean Maximum | 95% CI of |
| Chromosome | RS | QTA | Genotype | on Treatment | Genotype |
| position (bp) | Number | P-Value | (N) | ALTxULN (SE) | LS Mean |
| 6: 026202005 | rs2858996 | 0.0019 | G, G (74) | 0.06 (0.05) | (−0.03, 0.15) |
| | | | G, T (47) | 0.17 (0.06) | (0.06, 0.28) |
| | | | T, T (6) | 0.65 (0.16) | (0.33, 0.97) |

TABLE 4
Main Effect of HFE Genotypes from Case Control Analysis
of ALT Elevation in Whites, Second Study

| RS | FET | Genotype | Proportion of | Proportion of | Suspect | Odds Ratio |
|---|---|---|---|---|---|---|
| Number | P-Value | (N) | (N) Cases | (N) Controls | Genotype | (95% CI) |
| rs2858996 | 0.0034 | G, G (42) | 0.38 (8) | 0.63 (34) | T, T | 28.0 |
| | | | | | | (1.4, 546.9) |
| | | G, T (29) | 0.43 (9) | 0.37 (20) | | |
| | | T, T (4) | 0.19 (4) | 0.00 (0) | | |

The TT genotype of marker rs2858996 was associated with an increased risk of ALT elevation upon exposure to pazopanib in White subjects from both studies with an odds ratio for this risk genotype (with 95% confidence interval) being 15.5 (0.8, 301.0), and 28.0 (1.4, 546.9) respectively.

In the combined White subjects from the two studies, a statistically significant difference in the HFE (rs2858996) genotype distributions was observed between cases and controls (FET p=6.50×10). Twelve subjects had the TT genotype. Of these, 8 (67%) were ALT cases (ALT>3×ULN) and none were ALT controls (1×ULN or less). The remaining four subjects with the TT genotype had maximum ALT greater than 1×ULN and less than 3×ULN. These data predicted an odds ratio (95% CI) of 39.7 (2.24, 703.7) for cases versus controls, in relation to TT homozygotes versus the other genotypes (GG and GT genotypes).

Descriptive PGx analysis for the HFE marker and ALT in the other Race/Ethnicity groups from the two studies: 97 of the 99 non-White subjects were successfully genotyped for marker rs2858996. This collection of genotyped subjects consisted of 36 Hispanics, 47 Asians, 3 Blacks and 11 subjects having race/ethnicity other than White, Hispanic, Asian or Black. Only one subject had the TT genotype, and the maximum ALT value for this individual was within the normal range (i.e. less than 1×ULN). This subject was a self-reported Asian from the first study.

Descriptive PGx analysis for the HFE marker and ALT in another study: All 6 subjects were successfully genotyped for marker rs2858996. None of the subjects had the TT genotype.

Descriptive PGx analysis for the HFE marker and ALT in yet another study: All of the 22 subjects who were genotyped for marker rs2858996 and had ALT data available for PGx evaluation were successfully genotyped. Two subjects had the TT genotype; the maximum ALT values of the two subjects were within the normal range (i.e. less than 1×ULN). Both subjects were self-reported Whites.

Descriptive PGx analysis for the HFE marker and ALT in still another study: All of the 33 subjects in the PGx analysis populations were successfully genotyped for marker rs2858996. One individual had the TT genotype; this subject had the maximum ALT of 1.72×ULN and was a self-reported White.

Descriptive PGx analysis for the HFE marker and ALT in another study: All of the 24 subjects were successfully genotyped for marker rs2858996. None of the subjects had the TT genotype.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A method of prescribing a compound of formula (I) to a Caucasian patient in need thereof, said method comprising:
determining whether said patient has the TT genotype at the rs2858996 and/or rs707889 reference single nucleotide polymorphism; and
if said patient does not have the TT genotype at the rs2858996 and/or rs707889 reference single nucleotide polymorphism, prescribing to said patient a compound of formula (I): 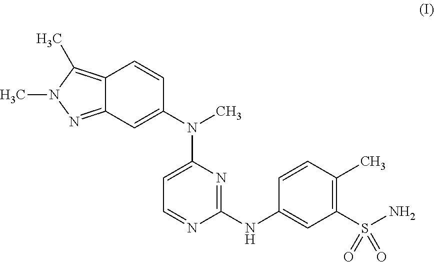
or a pharmaceutically acceptable salt or solvate thereof.

2. The method according to claim 1, wherein said prescribing comprises prescribing a compound of formula (I′):

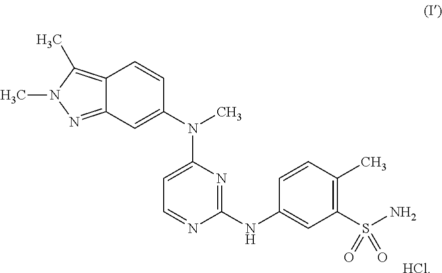

3. The method according to claim 1, wherein said prescribing comprises prescribing a compound of formula (I″):

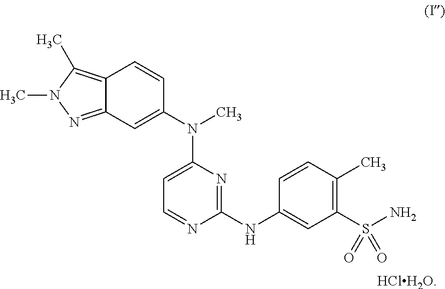

4. A method of administering a compound of formula (I) to a Caucasian patient in need thereof, said method comprising:
determining whether said patient has the TT genotype at the rs2858996 and/or rs707889 reference single nucleotide polymorphism; and
if said patient does not have the TT genotype at the rs2858996 and/or rs707889 reference single nucleotide polymorphism, administering to said patient a compound of formula (I): 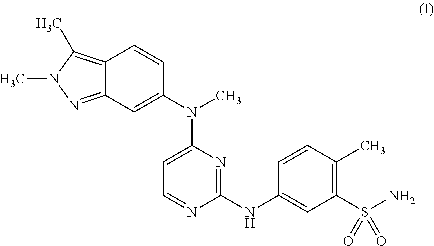
or a pharmaceutically acceptable salt or solvate thereof.

5. A method of treating cancer in a Caucasian patient in need thereof, said method comprising:
determining whether said patient has the TT genotype at the rs2858996 and/or rs707889 reference single nucleotide polymorphism; and
if said patient does not have the TT genotype at the rs2858996 and/or rs707889 reference single nucleotide polymorphism, administering to said patient a compound of formula 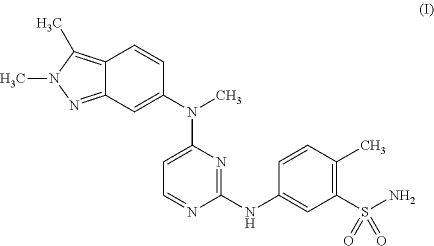
or a pharmaceutically acceptable salt or solvate thereof.

6. A method of treating cancer in a Caucasian patient in need thereof, said method comprising:
administering to said patient a compound of formula (I): 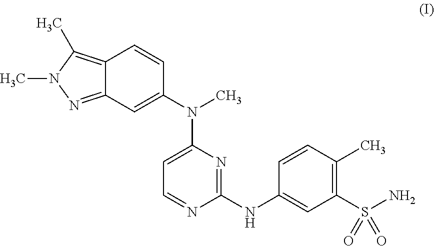
or a pharmaceutically acceptable salt or solvate thereof; and then
determining whether said patient has the TT genotype at the rs2858996 and/or rs707889 reference single nucleotide polymorphism.

7. A method of treating cancer in a Caucasian patient in need thereof, said method comprising:
administering to said patient a compound of formula (I): 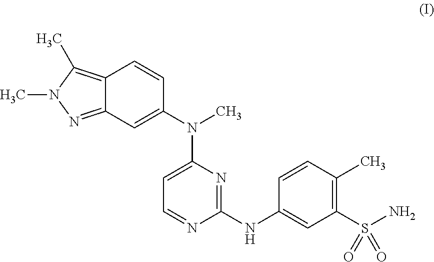
or a pharmaceutically acceptable salt or solvate thereof; and then
determining whether said patient has a significant elevation in alanine aminostransferase.

8. The method according to claim 7, further comprising if said patient has a significant elevation in alanine aminotransferase, determining whether said patient has the TT genotype at the rs2858996 and/or rs707889 reference single nucleotide polymorphism.

9. The method according to claim 7 or 8, further comprising discontinuing treatment with a compound according to formula (I), or a pharmaceutically acceptable salt or solvate thereof.

10. The method according to any one of claim 1, 4, 5, 6 or 8, wherein said determining comprises determining whether said patient has the TT genotype at the rs2858996 and rs707889 reference single nucleotide polymorphisms.

11. The method according to any one of claim 1, 4, 5, 6 or 8, wherein said determining comprises determining whether said patient has the TT genotype at rs2858996.

12. The method according to any one of claim 1, 4, 5, 6 or 8, wherein said determining comprises determining whether said patient has the TT genotype at rs707889.

13. The method according to any one of claim 1, 4, 5, 6 or 8, wherein said determining comprises testing said patient for the TT genotype at the rs2858996 and/or rs707889 reference single nucleotide polymorphism.

14. The method according to claim 13, wherein said determining comprises testing said patient for the TT genotype at the rs2858996 and rs707889 reference single nucleotide polymorphisms.

15. The method according to claim 13, wherein said determining comprises testing said patient for the TT genotype at the rs2858996 reference single nucleotide polymorphism.

16. The method according to claim 13, wherein said determining comprises testing said patient for the TT genotype at the rs707889 reference single nucleotide polymorphism.

17. The method according to any one of claim 1, 4, 5, 6 or 8, wherein said determining comprises testing said patient for at least one single nucleotide polymorphism that is correlated with the TT genotype of the rs2858996 and/or rs707889 reference single nucleotide polymorphism.

18. The method according to claim 17, wherein said determining comprises testing said patient for at least one single nucleotide polymorphism that is correlated with the TT genotype of the rs2858996 and rs707889 reference single nucleotide polymorphisms.

19. The method according to claim 17, wherein said determining comprises testing said patient for at least one single nucleotide polymorphism that is correlated with the TT genotype of the rs2858996 reference single nucleotide polymorphism.

20. The method according to claim 17, wherein said determining comprises testing said patient for at least one single nucleotide polymorphism that is correlated with the TT genotype of the rs707889 reference single nucleotide polymorphism.

21. A method of treating cancer in a Caucasian patient in need thereof, said patient genotyped as not having the TT genotype at the rs2858996 and/or rs707889 reference single nucleotide polymorphism, said method comprising:
administering to said patient a compound of Formula (I): 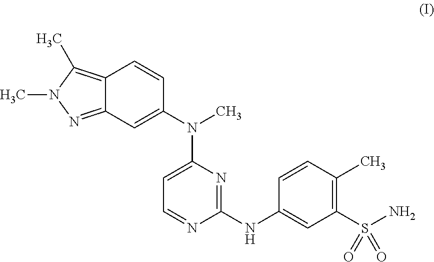
or a pharmaceutically acceptable salt or solvate thereof.

22. The method according to claim 21, said patient genotyped as not having the TT genotype at the rs2858996 and rs707889 reference single nucleotide polymorphisms.

23. The method according to claim 21, said patient genotyped as not having the TT genotype at rs2858996.

24. The method according to claim 21, said patient genotyped as not having the TT genotype at rs707889.

25. A method of treating cancer in a Caucasian patient in need thereof, said patient genotyped as not having at least one single nucleotide polymorphism that is correlated with the TT genotype of the rs2858996 and/or rs707889 reference single nucleotide polymorphism, said method comprising:
administering to said patient a compound of Formula (I): 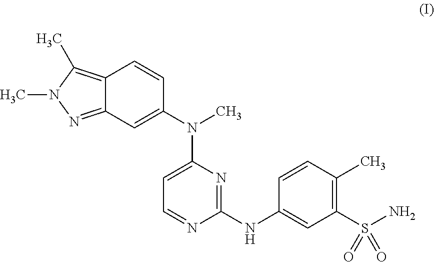
or a pharmaceutically acceptable salt or solvate thereof.

26. The method according to claim 25, said patient genotyped as not having at least one single nucleotide polymorphism that is correlated with the TT genotype of the rs2858996 and rs707889 reference single nucleotide polymorphisms.

27. The method according to claim 25, said patient genotyped as not having at least one single nucleotide polymorphism that is correlated with the TT genotype of the rs2858996 reference single nucleotide polymorphisms.

28. The method according to claim 25, said patient genotyped as not having at least one single nucleotide polymorphism that is correlated with the TT genotype of the rs707889 reference single nucleotide polymorphisms.

29. The method according to any one of claims 21 through 28, wherein said patient does not show significant elevation in alanine aminotransferase (ALT) after the administration of at least one dose of Formula I, or a pharmaceutically acceptable salt or solvate thereof.

30. A method of treating cancer in a Caucasian patient in need thereof, said method comprising:
determining whether said patient has the TT genotype at the rs2858996 and/or rs707889 reference single nucleotide polymorphism;
administering to said patient a compound of formula (I): 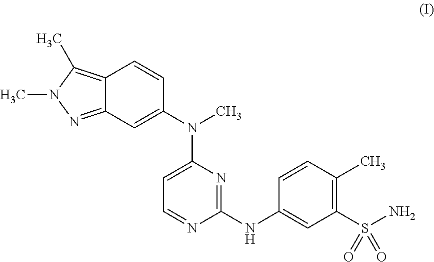
or a pharmaceutically acceptable salt or solvate thereof; and then
determining whether said patient has a significant elevation in alanine aminostransferase.

31. The method according to claim 30, wherein said determining whether said patient has the TT genotype at the rs2858996 and/or rs707889 reference single nucleotide polymorphism comprises determining whether said patient has the TT genotype as the rs2858996 and rs707889 reference single nucleotide polymorphisms.

32. The method according to claim 30, wherein said determining whether said patient has the TT genotype at the rs2858996 and/or rs707889 reference single nucleotide polymorphism comprises determining whether said patient has the TT genotype at rs2858996.

33. The method according to claim 30, wherein said determining whether said patient has the TT genotype at the rs2858996 and/or rs707889 reference single nucleotide polymorphism comprises determining whether said patient has the TT genotype at rs707889.

34. The method according to claim 30, wherein said determining whether said patient has the TT genotype at the rs2858996 and/or rs707889 reference single nucleotide polymorphism comprises testing said patient for the TT genotype at the rs2858996 and/or rs707889 reference single nucleotide polymorphism.

35. The method according to claim 30, wherein said determining whether said patient has the TT genotype at the rs2858996 and/or rs707889 reference single nucleotide polymorphism comprises testing said patient for the TT genotype at the rs2858996 and rs707889 reference single nucleotide polymorphisms.

36. The method according to claim 30, wherein said determining whether said patient has the TT genotype at the rs2858996 and/or rs707889 reference single nucleotide polymorphism comprises testing said patient for the TT genotype at the rs2858996 reference single nucleotide polymorphism.

37. The method according to claim 30, wherein said determining whether said patient has the TT genotype at the rs2858996 and/or rs707889 reference single nucleotide polymorphism comprises testing said patient for the TT genotype at the rs707889 reference single nucleotide polymorphism.

38. The method according to claim 30, wherein said determining whether said patient has the TT genotype at the rs2858996 and/or rs707889 reference single nucleotide polymorphism comprises testing said patient for at least one single nucleotide polymorphism that is correlated with the TT genotype of the rs2858996 and/or rs707889 reference single nucleotide polymorphism.

39. The method according to claim 30, wherein said determining whether said patient has the TT genotype at the rs2858996 and/or rs707889 reference single nucleotide polymorphism comprises testing said patient for at least one single nucleotide polymorphism that is correlated with the TT genotype of the rs2858996 and rs707889 reference single nucleotide polymorphisms.

40. The method according to claim 30, wherein said determining whether said patient has the TT genotype at the rs2858996 and/or rs707889 reference single nucleotide polymorphism comprises testing said patient for at least one single nucleotide polymorphism that is correlated with the TT genotype of the rs2858996 reference single nucleotide polymorphism.

41. The method according to claim 30, wherein said determining whether said patient has the TT genotype at the rs2858996 and/or rs707889 reference single nucleotide polymorphism comprises testing said patient for at least one single nucleotide polymorphism that is correlated with the TT genotype of the rs707889 reference single nucleotide polymorphism.

42. The method according to any one of claims 30 through 41, further comprising discontinuing treatment with a compound according to formula (I), or a pharmaceutically acceptable salt or solvate thereof.

43. The method according to any one of claims 5 through 42, wherein said cancer is selected from the group consisting of: colon cancer, breast cancer, renal cell carcinoma, melanoma, lung cancer including non-small cell lung cancer and adenocarcinoma, gastric cancer, colorectal cancer, neuroendocrine cancer, thyroid cancer, head and neck cancer, brain cancer, cervical cancer, bladder cancer, esophageal cancer, pancreatic cancer, prostate cancer, mesothelioma, liver-hepatobiliary cancer, multiple myeloma, leukemia, thyroid cancer including Hurthle cell, muscle sarcoma (leiomyosarcoma) and bone sarcoma (chonrosarcoma).

44. The method according to any one of claims 4 through 43, wherein the administration comprises administering a compound of formula (I′):

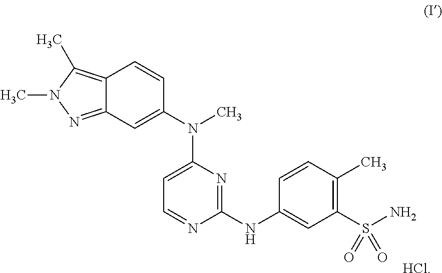

45. The method according to claim any one of claims 4 through 43, wherein said administration comprises administering a compound of formula (I″):

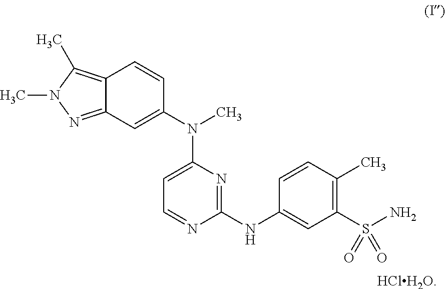

46. A method of screening a Caucasian human subject as an aid in predicting elevation in alanine aminotransferase (ALT) after administration of at least one dose of Formula I, or a pharmaceutically acceptable salt or solvate thereof, comprising:
determining whether said patient has the TT genotype at the rs2858996 and/or rs707889 reference single nucleotide polymorphism, wherein the presence of at least one TT genotype indicates the subject is at increased risk for increased ALT after administration of Formula I, or a pharmaceutically acceptable salt or solvate thereof.

47. A method of screening a Caucasian human subject as an aid in predicting elevation in alanine aminotransferase (ALT) after administration of at least one dose of Formula I, or a pharmaceutically acceptable salt or solvate thereof, comprising:
determining whether said patient has at least one single nucleotide polymorphism that is correlated with the TT genotype of the rs2858996 and/or rs707889 reference single nucleotide polymorphisms, wherein the presence of at least one single nucleotide polymorphism correlated with the TT genotype indicates the subject is at increased risk for increased ALT after administration of Formula I, or a pharmaceutically acceptable salt or solvate thereof.

48. A method of identifying a Caucasian human subject at increased risk of experiencing increased alanine aminotransferase (ALT) greater than or equal to three time upper limit normal after administration of at least one dose of Formula I, or a pharmaceutically acceptable salt or solvate thereof, comprising:
performing a genotyping technique on a biological sample from said subject to determine whether the subject has at least one single nucleotide polymorphism that is correlated with the TT genotype of the rs2858996 and/or rs707889;
detecting at least one single nucleotide polymorphism that is correlated with the TT genotype of the rs2858996 and/or rs707889; and
correlating the detection of at least one single nucleotide polymorphism that is correlated with the TT genotype of the rs2858996 and/or rs707889 with an increased risk of experiencing increased ALT greater than or equal to 3×ULN to at least one dose of Formula I, or a pharmaceutically acceptable salt thereof, compared to the risk if no single nucleotide polymorphism that is correlated with the TT genotype of the rs2858996 and/or rs707889 were detected.

49. A method according to claim 48, wherein said biological sample is selected from the group consisting of cells, blood, blood components, urine and saliva.


SEQUENCE LISTING (8 sequences)

SEQ ID NO: 1 - DNA, 53 bp, homo sapiens
ttactgtaccttaaccctgagtttgcgttagctatcactcaccaattatgcat

SEQ ID NO: 2 - DNA, 53 bp, homo sapien
gttccccttcatgtgattcaagctcacttcagaagaaacacaatgagacaaga

SEQ ID NO: 3 - DNA, 43 bp, Artificial Sequence
  5′ Oligonucleotide for detecting rs707889 polymorphism
acttcgtcagtaacggacgccccttcatgtgattcaagctcat

SEQ ID NO: 4 - DNA, 45 bp, Artificial Sequence
  5′ Oligonucleotide for detecting rs2858996 polymorphism
acttcgtcagtaacggacttactgtaccttaaccctgagtttgct

SEQ ID NO: 5 - DNA, 43 bp, Artificial Sequence
  5′ Oligonucleotide for detecting rs707889 polymorphism
gagtcgaggtcatatcgtgccccttcatgtgattcaagctcac

SEQ ID NO: 6 - DNA, 45 bp, Artificial Sequence
  5′ Oligonucleotide for detecting rs2858996 polymorphism
gagtcgaggtcatatcgtttactgtaccttaaccctgagtttgcg

SEQ ID NO: 7 - DNA, 63 bp, Artificial Sequence
  3′ Oligonucleotide for detecting rs707889 polymorphism
cagaagaaacacaatgagacaagacctacctgggacgtatggaacgtctgcctatagtgagtc

SEQ ID NO: 8 - DNA, 62 bp, Artificial Sequence
  3′ Oligonucleotide for detecting rs2858996 polymorphism
agctatcactcaccaattatgcacctacctgggacgtatggaacgtctgcctatagtgagtc